(19) United States
(12) Patent Application Publication
(10) Pub. No.: US 2025/0149123 A1
(43) Pub. Date: May 8, 2025

(54) METHODS, SYSTEMS, AND COMPUTER READABLE MEDIA FOR IMPROVING BASE CALLING ACCURACY

(71) Applicant: Life Technologies Corporation, Carlsbad, CA (US)

(72) Inventors: Ming Jiang, San Ramon, CA (US); Chengyong Yang, Foster City, CA (US); Jiaxin Zhang, Palo Alto, CA (US)

(21) Appl. No.: 19/016,754

(22) Filed: Jan. 10, 2025

Related U.S. Application Data
(63) Continuation of application No. 17/805,147, filed on Jun. 2, 2022.

(51) Int. Cl. G16B 40/20; G16B 30/00; G16B 40/10

(52) U.S. Cl. CPC G16B 40/20; G16B 30/00; G16B 40/10

(57) ABSTRACT

A method includes exposing template polynucleotide strands, sequencing primers, and polymerase to flows of nucleotide species; obtaining a series of measured intensity values and randomly selecting a training subset therefrom; generating series of base calls using a base caller and aligning the series of base calls to a reference genome using an aligner; determining intensity value thresholds and parameters of a linear transformation corresponding to different homopolymer lengths and nucleotide species; generating series of base calls corresponding to the series of measured intensity values using at least some of the parameters of a linear transformation; and recalibrating the series of base calls corresponding to the plurality of series of measured intensity values using at least some of the intensity value thresholds.

### CROSS-REFERENCE TO RELATED APPLICATIONS

This application is a Continuation of U.S. application Ser. No. 17/805,147 filed Jun. 2, 2022, which is a Continuation of U.S. application Ser. No. 16/055,315 filed Aug. 6, 2018, which is a Division of U.S. application Ser. No. 14/255,528 filed Apr. 17, 2014, which claims priority to U.S. application No. 61/879,910 filed Sep. 19, 2013, and to U.S. application No. 61/814,061 filed Apr. 19, 2013, which disclosures are herein incorporated by reference in their entirety.

### FIELD

This application generally relates to methods, systems, and computer readable media for nucleic acid sequencing, and, more specifically, to methods, systems, and computer readable media for improving base calling accuracy when sequencing nucleic acid sequencing data.

### BACKGROUND

Nucleic acid sequencing data may be obtained in various ways, including using next-generation sequencing systems such as, for example, the Ion PGM™ and Ion Proton™ systems implementing Ion Torrent™ sequencing technology; see, e.g., U.S. Pat. No. 7,948,015 and U.S. Pat. Appl. Publ. Nos. 2010/0137143, 2009/0026082, and 2010/0282617, which are all incorporated by reference herein in their entirety. There is a need for new methods, systems, and computer readable media that can better evaluate base calls and reduce sequencing errors when analyzing data obtained using these or other sequencing systems/platforms.

### BRIEF DESCRIPTION OF THE DRAWINGS

The accompanying drawings, which are incorporated into and form a part of the specification, illustrate one or more exemplary embodiments and serve to explain the principles of various exemplary embodiments. The drawings are exemplary and explanatory only and are not to be construed as limiting or restrictive in any way.

FIG. 1 illustrates an exemplary system for improving base calling accuracy.

FIG. 2 illustrates exemplary components of an apparatus for nucleic acid sequencing.

FIG. 3 illustrates an exemplary flow cell for nucleic acid sequencing.

FIG. 4 illustrates an exemplary process for label-free, pH-based sequencing.

FIG. 5 illustrates an exemplary computer system.

FIG. 6 illustrates an exemplary method for improving base calling accuracy.

FIG. 7 illustrates an exemplary method for determining recalibration thresholds and model parameters.

FIG. 8 illustrates a plot of accuracy as a function of intensities showing examples of recalibration thresholds.

FIGS. 9A-9D illustrate exemplary plots of measurement/prediction clusters.

FIGS. 10A-10D illustrate exemplary plots of measurement/prediction clusters.

FIGS. 11A-11D illustrate exemplary plots of measurement/prediction clusters.

FIG. 12 shows a table with an example of recalibration model parameters.

FIG. 13 illustrates an exemplary method for recalibrating sequencing data.

FIGS. 14A and 14B illustrate exemplary plots of error rate as a function of homopolymer length before and after recalibration.

### SUMMARY

According to an exemplary embodiment, there is provided a method for improving base calling accuracy in nucleic acid sequencing, comprising: (a) exposing a plurality of template polynucleotide strands, sequencing primers, and polymerase disposed in a plurality of defined spaces disposed on a sensor array to a series of flows of nucleotide species according to a predetermined order; (b) obtaining a plurality of series of measured intensity values corresponding to the series of flows of nucleotide species and to the plurality of defined spaces disposed on the sensor array and randomly selecting a training subset of the plurality of series of measured intensity values; (c) generating a first plurality of series of base calls corresponding to the training subset of the plurality of series of measured intensity values using a base caller and aligning the first plurality of series of base calls to a reference genome or sequence using an aligner; (d) determining a plurality of intensity value thresholds corresponding to different homopolymer lengths and nucleotide species, and a plurality of parameters of a linear transformation corresponding to different homopolymer lengths and nucleotide species; (e) generating a second plurality of series of base calls corresponding to the plurality of series of measured intensity values using the base caller and, for homopolymers of a least a first predetermined length, at least some of the plurality of parameters of a linear transformation; and (f) recalibrating the second plurality of series of base calls corresponding to the plurality of series of measured intensity values, for homopolymers of at most a second predetermined length, using at least some of the plurality of intensity value thresholds.

According to an exemplary embodiment, there is provided a non-transitory machine-readable storage medium comprising instructions which, when executed by a processor, cause the processor to perform a method for improving base calling accuracy in nucleic acid sequencing, comprising: (a) exposing a plurality of template polynucleotide strands, sequencing primers, and polymerase disposed in a plurality of defined spaces disposed on a sensor array to a series of flows of nucleotide species according to a predetermined order; (b) obtaining a plurality of series of measured intensity values corresponding to the series of flows of nucleotide species and to the plurality of defined spaces disposed on the sensor array and randomly selecting a training subset of the plurality of series of measured intensity values; (c) generating a first plurality of series of base calls corresponding to the training subset of the plurality of series of measured intensity values using a base caller and aligning the first plurality of series of base calls to a reference genome or sequence using an aligner; (d) determining a plurality of intensity value thresholds corresponding to different homopolymer lengths and nucleotide species, and a plurality of parameters of a linear transformation corresponding to different homopolymer lengths and nucleotide species; (c) generating a second plurality of series of base calls corresponding to the plurality of series of measured intensity values using the base caller and, for homopolymers of a least a first predetermined length, at least some of the plurality of parameters of a linear transformation; and (f) recalibrating the second plurality of series of base calls corresponding to the plurality of series of measured intensity values, for homopolymers of at most a second predetermined length, using at least some of the plurality of intensity value thresholds.

According to an exemplary embodiment, there is provided a system for improving base calling accuracy in nucleic acid sequencing, including: a plurality of template polynucleotide strands, sequencing primers, and polymerase disposed in a plurality of defined spaces disposed on a sensor array; an apparatus configured to expose the plurality of template polynucleotide strands, sequencing primers, and polymerase to a series of flows of nucleotide species according to a predetermined order; a machine-readable memory; and a processor configured to execute machine-readable instructions, which, when executed by the processor, cause the system to perform a method for improving base calling accuracy in nucleic acid sequencing, comprising: (a) obtaining a plurality of series of measured intensity values corresponding to the series of flows of nucleotide species and to the plurality of defined spaces disposed on the sensor array and randomly selecting a training subset of the plurality of series of measured intensity values; (b) generating a first plurality of series of base calls corresponding to the training subset of the plurality of series of measured intensity values using a base caller and aligning the first plurality of series of base calls to a reference genome or sequence using an aligner; (c) determining a plurality of intensity value thresholds corresponding to different homopolymer lengths and nucleotide species, and a plurality of parameters of a linear transformation corresponding to different homopolymer lengths and nucleotide species; (d) generating a second plurality of series of base calls corresponding to the plurality of series of measured intensity values using the base caller and, for homopolymers of a least a first predetermined length, at least some of the plurality of parameters of a linear transformation; and (e) recalibrating the second plurality of series of base calls corresponding to the plurality of series of measured intensity values, for homopolymers of at most a second predetermined length, using at least some of the plurality of intensity value thresholds.

### EXEMPLARY EMBODIMENTS

The following description and the various embodiments described herein are exemplary and explanatory only and are not to be construed as limiting or restrictive in any way. Other embodiments, features, objects, and advantages of the present teachings will be apparent from the description and accompanying drawings, and from the claims.

According to various exemplary embodiments, methods, systems, and computer readable media for improving base calling accuracy in nucleic acid sequencing using recalibration of base calls or related intensity signals or parameters are disclosed herein. The various embodiments may improve accuracy by performing recalibration of base calls or related intensity signals or parameters using a training subset of called reads that were aligned to a reference genome or sequence, which may compensate for systematic bias that may be present in nucleic acid sequencing signals and often results in under-calls or over-calls. Such methods, systems, and computer readable media may reduce certain systematic errors and improve overall sequencing accuracy (especially in the case of long homopolymers), which may in turn improve downstream processing such as variant calling.

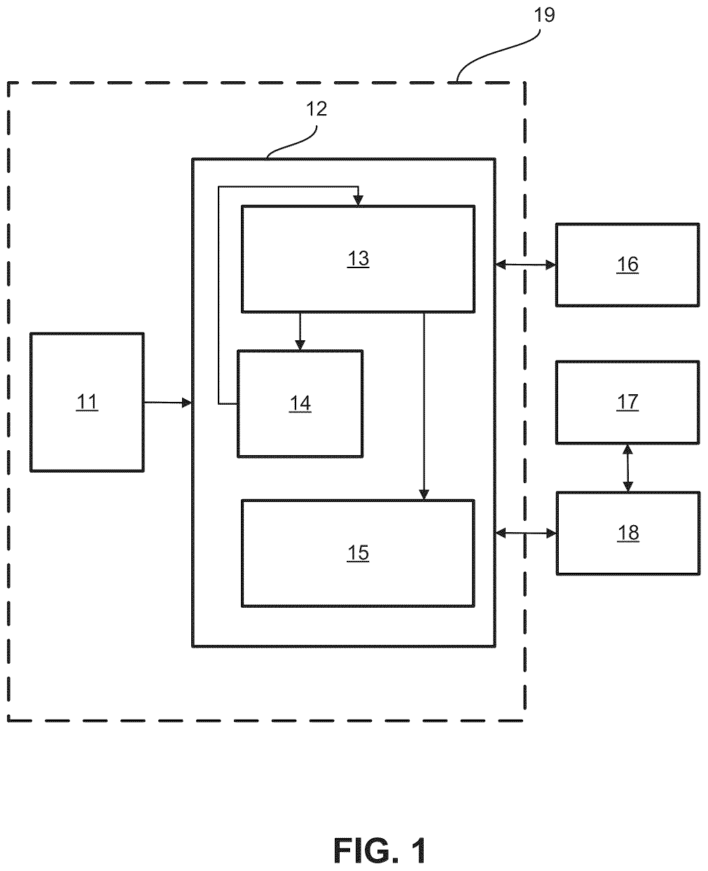

FIG. 1 illustrates an exemplary system for improving base calling accuracy. The system includes an apparatus or sub-system for nucleic acid sequencing and/or analysis 11, a computing server/node/device 12 including a base calling engine 13, a recalibration engine 14, a post-processing engine 15, and a display 16, which may be internal and/or external. The apparatus or sub-system for nucleic acid sequencing and/or analysis 11 may be any type of instrument that can generate nucleic acid sequence data from nucleic acid samples, which may include a nucleic acid sequencing instrument, a real-time/digital/quantitative PCR instrument, a microarray scanner, etc. The computing server/node/device 12 may be a workstation, mainframe computer, distributed computing node (part of a “cloud computing” or distributed networking system), personal computer, mobile device, etc. The base calling engine 13 may be configured to include various signal/data processing modules that may be configured to receive signal/data from the apparatus or sub-system for nucleic acid sequencing and/or analysis 11 and perform various processing steps, such as conversion from flow space to base space, determination of base calls for some or the entirety of a sequencing data set, and determination of base call quality values. In an embodiment, the base calling engine 13 may implement one or more features described in Davey et al., U.S. Pat. Appl. Publ. No. 2012/0109598, published on May 3, 2012, and/or Sikora et al., U.S. Pat. Appl. Publ. No. 2013/0060482, published on Mar. 7, 2012, which are all incorporated by reference herein in their entirety. The base calling engine 13 may also include a mapping or alignment module for mapping or aligning reads to a reference sequence or genome, which may be a whole/partial genome, whole/partial exome, etc. In an embodiment, the mapping or alignment module may include any suitable aligner, including the Torrent Mapping Alignment Program (TMAP), for example. The recalibration engine 14 may be configured to recalibrate base calls or related intensity values or parameters based on an analysis of base calling and alignment performed by the base calling engine 13, which recalibrated base calls or related intensity values or thresholds or parameters may be fed back into the base calling engine 13 for improving the accuracy of base calls. The recalibration engine 14 may also include both a non-parametric recalibration module and a parametric model-based recalibration module. The exemplary system may also include a client device terminal 17, which may include a data analysis API or module and may be communicatively connected to the computing server/node/device 12 via a network connection 18 that may be a “hardwired” physical network connection (e.g., Internet, LAN, WAN, VPN, etc.) or a wireless network connection (e.g., Wi-Fi, WLAN, etc.). The post-processing engine 15 may be configured to include various signal/data processing modules that may be configured to make variant calls and apply post-processing to variant calls, which may include annotating various variant calls and/or features, converting data from flow space to base space, filtering of variants, and formatting the variant data for display or use by client device terminal 17. In an embodiment, the apparatus or sub-system for nucleic acid sequencing and/or analysis 11 and the computing server/node/device 12 may be integrated into a single instrument or system comprising components present in a single enclosure 19. The client device terminal 17 may be configured to communicate information to and/or control the operation of the computing server/node/device 12 and its modules and/or operating parameters.

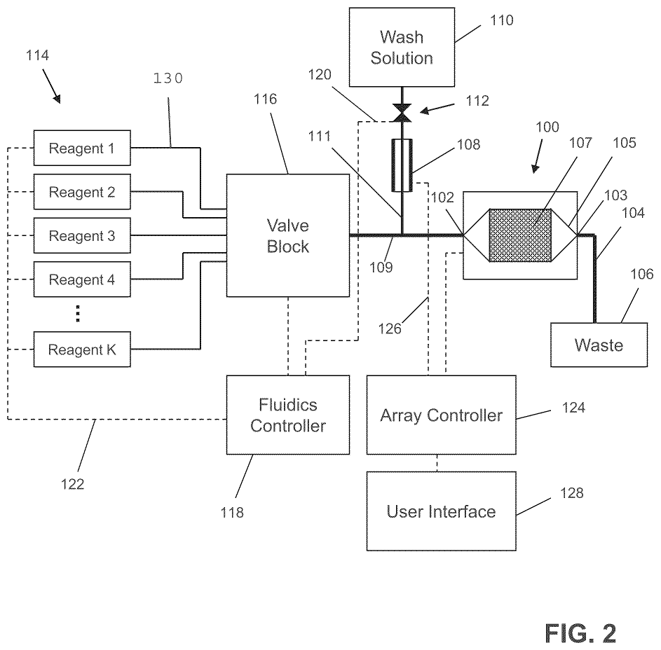

FIG. 2 illustrates exemplary components of an apparatus for nucleic acid sequencing. Such an apparatus could be used as apparatus or sub-system for nucleic acid sequencing and/or analysis 11 of FIG. 1. The components include a flow cell and sensor array 100, a reference electrode 108, a plurality of reagents 114, a valve block 116, a wash solution 110, a valve 112, a fluidics controller 118, lines 120/122/126, passages 104/109/111, a waste container 106, an array controller 124, and a user interface 128. The flow cell and sensor array 100 includes an inlet 102, an outlet 103, a microwell array 107, and a flow chamber 105 defining a flow path of reagents over the microwell array 107. The reference electrode 108 may be of any suitable type or shape, including a concentric cylinder with a fluid passage or a wire inserted into a lumen of passage 111. The reagents 114 may be driven through the fluid pathways, valves, and flow cell by pumps, gas pressure, or other suitable methods, and may be discarded into the waste container 106 after exiting the flow cell and sensor array 100. The reagents 114 may, for example, contain dNTPs to be flowed through passages 130 and through the valve block 116, which may control the flow of the reagents 114 to flow chamber 105 (also referred to herein as a reaction chamber) via passage 109. The system may include a reservoir 110 for containing a wash solution that may be used to wash away dNTPs, for example, that may have previously been flowed. The microwell array 107 may include an array of defined spaces, such as microwells, for example, that is operationally associated with a sensor array so that, for example, each microwell has a sensor suitable for detecting an analyte or reaction property of interest. The microwell array 107 may preferably be integrated with the sensor array as a single device or chip. The array controller 124 may provide bias voltages and timing and control signals to the sensor, and collect and/or process output signals. The user interface 128 may display information from the flow cell and sensor array 100 as well as instrument settings and controls, and allow a user to enter or set instrument settings and controls. The valve 112 may be shut to prevent any wash solution 110 from flowing into passage 109 as the reagents are flowing. Although the flow of wash solution may be stopped, there may still be uninterrupted fluid and electrical communication between the reference electrode 108, passage 109, and the sensor array 107. The distance between the reference electrode 108 and the junction between passages 109 and 111 may be selected so that little or no amount of the reagents flowing in passage 109 and possibly diffusing into passage 111 reach the reference electrode 108. In various embodiments, the fluidics controller 118 may be programmed to control driving forces for flowing reagents 114 and the operation of valve 112 and valve block 116 to deliver reagents to the flow cell and sensor array 100 according to a predetermined reagent flow ordering.

In this application, “defined space” generally refers to any space (which may be in one, two, or three dimensions) in which at least some of a molecule, fluid, and/or solid can be confined, retained and/or localized. The space may be a predetermined area (which may be a flat area) or volume, and may be defined, for example, by a depression or a micro-machined well in or associated with a microwell plate, microtiter plate, microplate, or a chip, or by isolated hydrophobic areas on a generally hydrophobic surface. Defined spaces may be arranged as an array, which may be a substantially planar one-dimensional or two-dimensional arrangement of elements such as sensors or wells. Defined spaces, whether arranged as an array or in some other configuration, may be in electrical communication with at least one sensor to allow detection or measurement of one or more detectable or measurable parameter or characteristics. The sensors may convert changes in the presence, concentration, or amounts of reaction by-products (or changes in ionic character of reactants) into an output signal, which may be registered electronically, for example, as a change in a voltage level or a current level which, in turn, may be processed to extract information or signal about a chemical reaction or desired association event, for example, a nucleotide incorporation event and/or a related ion concentration (e.g., a pH measurement). The sensors may include at least one ion sensitive field effect transistor (“ISFET”) or chemically sensitive field effect transistor (“chemFET”).

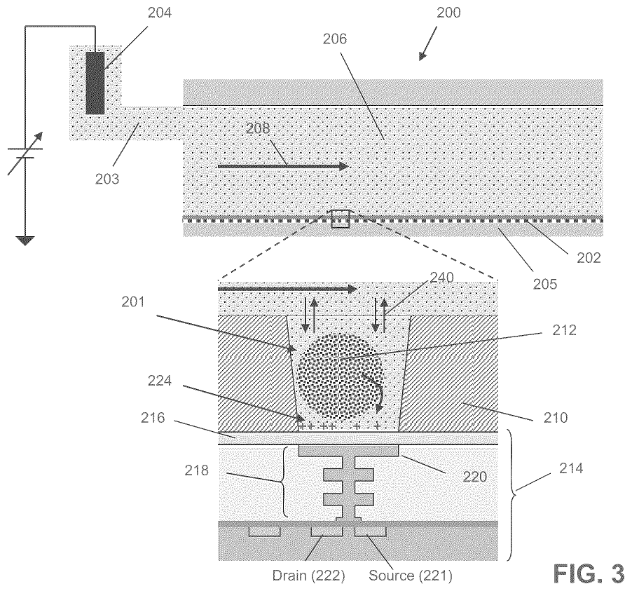

FIG. 3 illustrates an exemplary flow cell for nucleic acid sequencing. The flow cell 200 includes a microwell array 202, a sensor array 205, and a flow chamber 206 in which a reagent flow 208 may move across a surface of the microwell array 202, over open ends of microwells in the microwell array 202. The flow of reagents (e.g., nucleotide species) can be provided in any suitable manner, including delivery by pipettes, or through tubes or passages connected to a flow chamber. A microwell 201 in the microwell array 202 may have any suitable volume, shape, and aspect ratio. A sensor 214 in the sensor array 205 may be an ISFET or a chemFET sensor with a floating gate 218 having a sensor plate 220 separated from the microwell interior by a passivation layer 216, and may be predominantly responsive to (and generate an output signal related to) an amount of charge 224 present on the passivation layer 216 opposite of the sensor plate 220. Changes in the amount of charge 224 cause changes in the current between a source 221 and a drain 222 of the sensor 214, which may be used directly to provide a current-based output signal or indirectly with additional circuitry to provide a voltage output signal. Reactants, wash solutions, and other reagents may move into microwells primarily by diffusion 240. One or more analytical reactions to identify or determine characteristics or properties of an analyte of interest may be carried out in one or more microwells of the microwell array 202. Such reactions may generate directly or indirectly by-products that affect the amount of charge 224 adjacent to the sensor plate 220. In an embodiment, a reference electrode 204 may be fluidly connected to the flow chamber 206 via a flow passage 203. In an embodiment, the microwell array 202 and the sensor array 205 may together form an integrated unit forming a bottom wall or floor of the flow cell 200. In an embodiment, one or more copies of an analyte may be attached to a solid phase support 212, which may include microparticles, nanoparticles, beads, gels, and may be solid and porous, for example. The analyte may include one or more copies of a nucleic acid analyte obtained using any suitable technique.

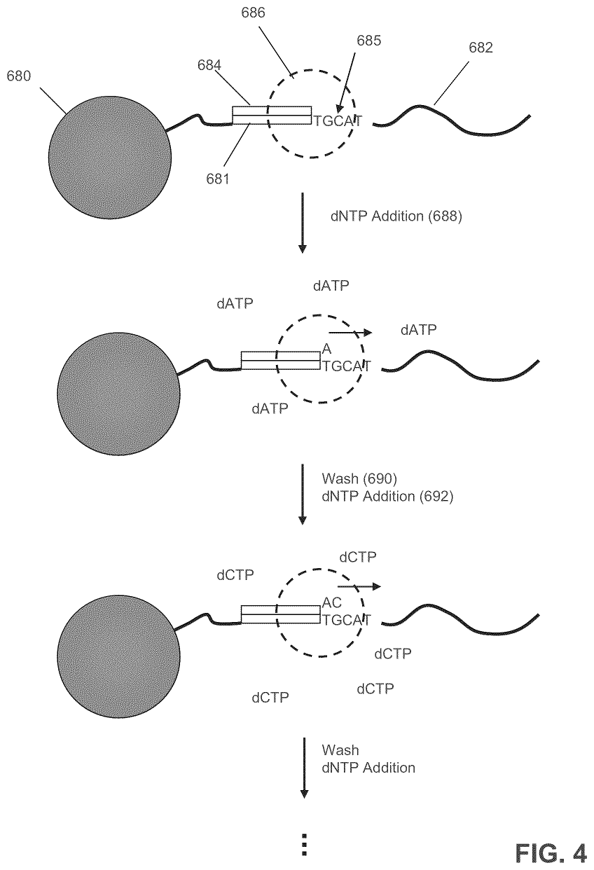

FIG. 4 illustrates an exemplary process for label-free, pH-based sequencing. A template 682 with sequence 685 and a primer binding site 681 are attached to a solid phase support 680. The template 682 may be attached as a clonal population to a solid support, such as a microparticle or bead, for example, and may be prepared as disclosed in Leamon et al., U.S. Pat. No. 7,323,305. In an embodiment, the template may be associated with a substrate surface or present in a liquid phase with or without being coupled to a support. A primer 684 and DNA polymerase 686 are annealed to the template 682 so that the primer's 3′ end may be extended by a polymerase and that a polymerase is bound to such primer-template duplex (or in close proximity thereof) so that binding and/or extension may take place when dNTPs are added. In step 688, dNTP (shown as dATP) is added, and the DNA polymerase 686 incorporates a nucleotide “A” (since “T” is the next nucleotide in the template 682 and is complementary to the flowed dATP nucleotide). In step 690, a wash is performed. In step 692, the next dNTP (shown as dCTP) is added, and the DNA polymerase 686 incorporates a nucleotide “C” (since “G” is the next nucleotide in the template 682). More details about pH-based nucleic acid sequencing may be found in U.S. Pat. No. 7,948,015 and U.S. Pat. Appl. Publ. Nos. 2010/0137143, 2009/0026082, and 2010/0282617.

In an embodiment, the primer-template-polymerase complex may be subjected to a series of exposures of different nucleotides in a pre-determined sequence or ordering. If one or more nucleotides are incorporated, then the signal resulting from the incorporation reaction may be detected, and after repeated cycles of nucleotide addition, primer extension, and signal acquisition, the nucleotide sequence of the template strand may be determined. The output signals measured throughout this process depend on the number of nucleotide incorporations. Specifically, in each addition step, the polymerase extends the primer by incorporating added dNTP only if the next base in the template is complementary to the added dNTP. With each incorporation, an hydrogen ion is released, and collectively a population released hydrogen ions change the local pH of the reaction chamber. The production of hydrogen ions may be monotonically related to the number of contiguous complementary bases (e.g., homopolymers) in the template. Deliveries of nucleotides to a reaction vessel or chamber may be referred to as “flows” of nucleotide triphosphates (or dNTPs). For convenience, a flow of dATP will sometimes be referred to as “a flow of A” or “an A flow,” and a sequence of flows may be represented as a sequence of letters, such as “ATGT” indicating “a flow of dATP, followed by a flow of dTTP, followed by a flow of dGTP, followed by a flow of dTTP.” The predetermined ordering may be based on a cyclical, repeating pattern consisting of consecutive repeats of a short pre-determined reagent flow ordering (e.g., consecutive repeats of pre-determined sequence of four nucleotide reagents such as, for example, “ACTG ACTG . . . ”), may be based in whole or in part on some other pattern of reagent flows (such as, e.g., any of the various reagent flow orderings discussed herein and/or in Hubbell et al., U.S. Pat. Appl. Publ. No. 2012/0264621, published Oct. 18, 2012, which is incorporated by reference herein in its entirety), and may also be based on some combination thereof.

In various embodiments, output signals due to nucleotide incorporation may be processed, given knowledge of what nucleotide species were flowed and in what order to obtain such signals, to make base calls for the flows and compile consecutive base calls associated with a sample nucleic acid template into a read. A base call refers to a particular nucleotide identification (e.g., dATP (“ A”), dCTP (“C”), dGTP (“G”), or dTTP (“T”)). Base calling may include performing one or more signal normalizations, signal phase and signal decay (e.g., enzyme efficiency loss) estimations, signal corrections, and model-based signal predictions, and may identify or estimate base calls for each flow for each defined space. Any suitable base calling method may be used, including as described in Davey et al., U.S. Pat. Appl. Publ. No. 2012/0109598, published on May 3, 2012, and/or Sikora et al., U.S. Pat. Appl. Publ. No. 2013/0060482, published on Mar. 7, 2012, which are all incorporated by reference herein in their entirety, recognizing of course that more accurate base callers may yield better results.

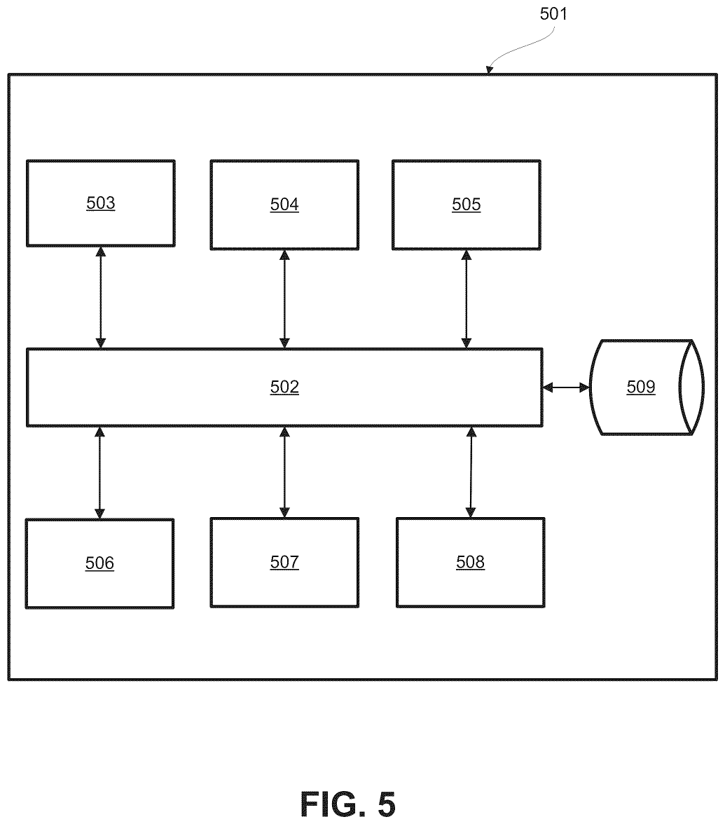

FIG. 5 illustrates an exemplary computer system. Such a computer system could be used as computing server/node/device 12 of FIG. 1. The computer system 501 includes a bus 502 or other communication mechanism for communicating information, a processor 503 coupled to the bus 502 for processing information, and a memory 505 coupled to the bus 502 for dynamically and/or statically storing information. The computer system 501 can also include one or more co-processors 504 coupled to the bus 502, such as GPUs and/or FPGAs, for performing specialized processing tasks; a display 506 coupled to the bus 502, such as a cathode ray tube (CRT) or liquid crystal display (LCD), for displaying information to a computer user; an input device 507 coupled to the bus 502, such as a keyboard including alphanumeric and other keys, for communicating information and command selections to the processor 503; a cursor control device 508 coupled to the bus 502, such as a mouse, a trackball or cursor direction keys for communicating direction information and command selections to the processor 503 and for controlling cursor movement on display 506; and one or more storage devices 509 coupled to the bus 502, such as a magnetic disk or an optical disk, for storing information and instructions. The memory 505 may include a random access memory (RAM) or other dynamic storage device and/or a read only memory (ROM) or other static storage device. Such an exemplary computer system with suitable software may be used to perform the embodiments described herein. More generally, in various embodiments, one or more features of the teachings and/or embodiments described herein may be performed or implemented using appropriately configured and/or programmed hardware and/or software elements.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various embodiments, one or more features of teachings and/or embodiments described herein may be performed or implemented using an appropriately configured and/or programmed non-transitory machine-readable medium or article that may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Flash memory, hard drive, tape, CDROM, etc.). Moreover, memory can incorporate electronic, magnetic, optical, and/or other types of storage media. Memory can have a distributed, clustered, remote, or cloud architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

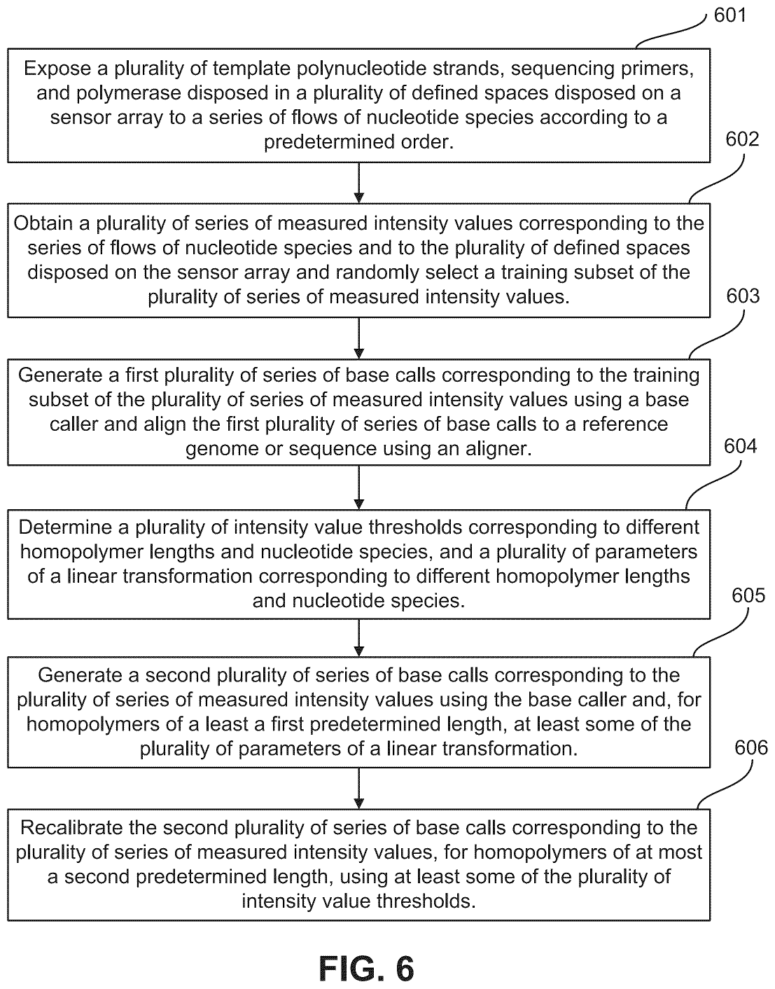

FIG. 6 illustrates an exemplary method for improving base calling accuracy. In step 601, a user or component exposes a plurality of template polynucleotide strands, sequencing primers, and polymerase disposed in a plurality of defined spaces or regions disposed on a sensor array to a series of flows of nucleotide species according to a predetermined order. In step 602, a user or component obtains a plurality of series of measured intensity values corresponding to the series of flows of nucleotide species and to the plurality of defined spaces disposed on the sensor array and randomly selects a training subset of the plurality of series of measured intensity values. In step 603, a server or other computing means or resource generates a first plurality of series of base calls corresponding to the training subset of the plurality of series of measured intensity values using a base caller and aligns the first plurality of series of base calls to a reference genome or sequence using an aligner. The measured intensity values may be related to voltage data indicative of hydrogen ion concentrations representative of a number of nucleotide incorporations or may include any other type of data (e.g., pyrophosphate, light, etc.) that could be representative of a number of nucleotide incorporations. In step 604, the server or other computing means or resource determines a plurality of intensity value thresholds corresponding to different homopolymer lengths and nucleotide species, and a plurality of parameters of a linear transformation corresponding to different homopolymer lengths and nucleotide species. In step 605, the server or other computing means or resource generates a second plurality of series of base calls corresponding to the plurality of series of measured intensity values using the base caller and, for homopolymers of a least a first predetermined length, at least some of the plurality of parameters of a linear transformation. In step 606, the server or other computing means or resource recalibrates the second plurality of series of base calls corresponding to the plurality of series of measured intensity values, for homopolymers of at most a second predetermined length, using at least some of the plurality of intensity value thresholds. Such recalibrated base calls may generally improve accuracy and reduce or compensate for systematic bias.

In various embodiments, recalibration methods as discussed herein may be performed in parallel on subdivisions or partitions of a sensor array or chip. For example, a sensor array or chip may be divided into two or more regions (which could be physical regions, such as quadrants, or could be based on a content of regions or portions, such as library fragments vs. test fragments, for example) and recalibration may be performed on each region independently of the others. In some cases, recalibration may also be performed separately for different groups of nucleotide flows (e.g., earlier flows vs. later flows).

In various embodiments, recalibration methods as discussed herein may be performed in two stages: (i) a training stage, and (ii) a run stage. In the training stage, one or more recalibration thresholds and model parameters may be determined using a training set of base sequences aligned to a reference genome or sequence. In the run stage, the one or more recalibration thresholds and model parameters may be used to recalibrate base calls or related intensity signals or parameters to improving base calling accuracy.

In various embodiments, recalibration methods as discussed herein may comprise both a non-parametric recalibration module and a parametric model-based recalibration module, which can yield more accurate base calling without significant impact on computation efficiency. In various embodiments, one or two decision points or configurable switches may be used to control which one(s) of the approaches are used for homopolymers of a given length. In some cases, non-parametric recalibration may be used for homopolymers of at most a given length and parametric model-based recalibration may be used for homopolymers exceeding that given length (possibly up to some preset maximum length). In other cases, there may be some overlap, wherein non-parametric recalibration may be used for homopolymers of at most a first given length and parametric model-based recalibration may be used for homopolymers of at least a second given length (possibly up to some preset maximum length).

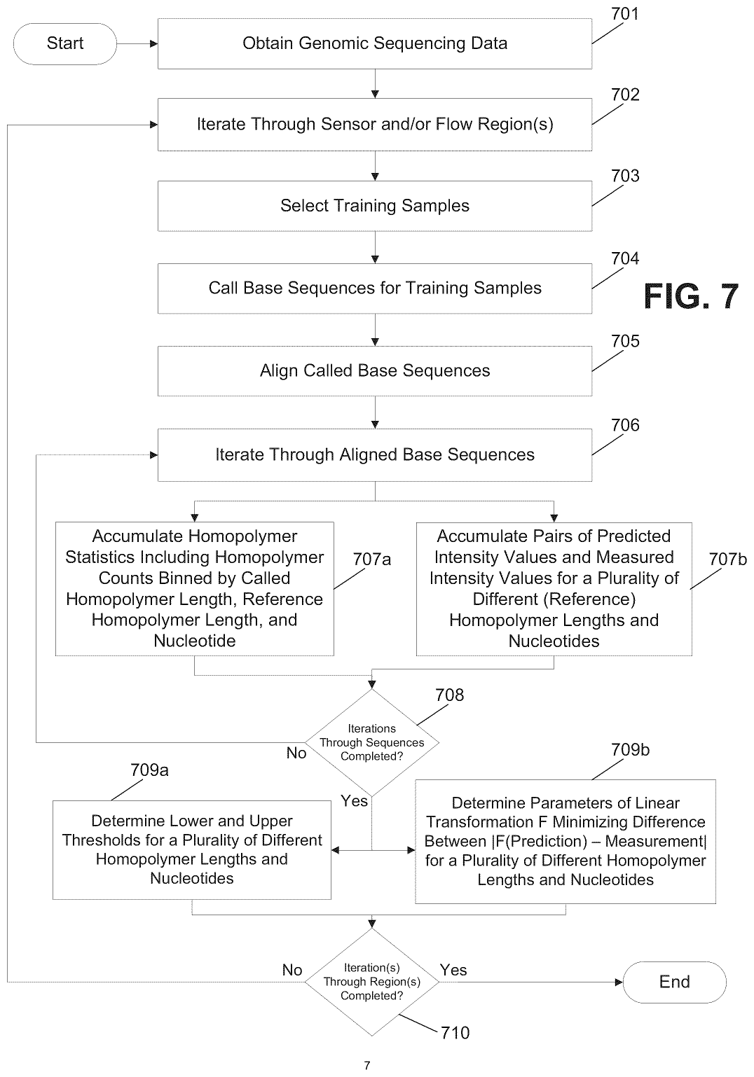

FIG. 7 illustrates an exemplary method for determining recalibration thresholds and model parameters. In step 701, a user or component obtains genomic sequencing data, which may be any kind of sequencing data that can be used to obtain base calls, including data from a plurality of defined spaces in a sensor array configured to detect nucleotide incorporations. The data may include, e.g., measured intensity values that may be related to voltage data indicative of hydrogen ion concentrations representative of a number of nucleotide incorporations or may include any other type of data (e.g., pyrophosphate, light, etc.) that could otherwise be representative of a number of nucleotide incorporations. In step 702, a server or other computing means or resource iterates through one or more sensor region and/or flow region. In some cases, the sensor may have a single region, and in other cases it may have multiple distinct regions (e.g., 2, 3, 4, or more). In some cases, the flows may be viewed as a single group of flows (e.g., a group of 250 flows, of 500 flows, or more), and in other cases the flows may be viewed as multiple groups of flows (e.g., a first group of 250 flows and a second group of 250 flows following the first group). In step 703, the server or other computing means or resource selects training samples in the one or more sensor region and/or flow region, which may be done separately for every region, or all at once with the subset then being divided across the regions if multiple regions are present. Any suitable method for sample selection/sampling may be used to select the training set, including random selection. In step 704, the server or other computing means or resource calls base sequences for the training samples using a base caller, which may be any suitable base caller, including preferably a base caller based at least in part on predictive modeling, as further discussed below. In step 705, the server or other computing means or resource aligns or maps the called base sequences to a reference genome or sequence, which may be done using any suitable aligner or mapper known in the art, which may be the Torrent Mapping Alignment Program (TMAP), for example. The aligned or mapped sequences may contain or be associated with information from both the base caller and alignment/mapping, which information may include one or more of the following (depending on the base caller and underlying sequencing technology): a called sequence, a reference sequence, (normalized) measurement intensity values (e.g., signal), lower/upper homopolymer boundary perturbations (e.g., a deviation from an idealized threshold measurement intensity value), and one or more base calling parameters (e.g., phasing rate, decay, etc.). In step 706, the server or other computing means or resource iterates through the aligned or mapped base sequences. In step 707a, the server or other computing means or resource accumulates (e.g., consolidates and processes) homopolymer statistics including homopolymer counts binned by called homopolymer length, called reference homopolymer length, and nucleotide. The statistics may be obtained from the sequence either in base-space representation or in flow-space representation. In step 707b, the server or other computing means or resource accumulates pairs of predicted intensity values (e.g., via a predictive model used by the base caller) and measured intensity values for a plurality of different homopolymer lengths and nucleotides (e.g., homopolymers of length 1 through 4, or 1 through 6, or 1 through 8, or more, and nucleotides A, C, G, and T). In step 708, the server or other computing means or resource repeats steps 707a and 707b until iterations through the aligned base sequences have been completed. In step 709a, the server or other computing means or resource determines lower and upper thresholds for a plurality of different homopolymer lengths and nucleotides using a graph of accuracy as a function of intensity value, which graph may be generated using the homopolymer statistics accumulated in step 707a and as further discussed below. In step 709b, the server or other computing means or resource determines parameters of a linear function F minimizing |F(prediction)−measurement| for a plurality of different homopolymer lengths and nucleotides using some or all of the pairs accumulated in step 707b. In step 710, the server or other computing means or resource repeats steps 702 through 709a and 709b until iterations through the sensor and/or flow region(s) have been completed.

### Non-Parametric Recalibration

In an embodiment, the homopolymer statistics may be accumulated as follows to determine the lower and upper thresholds for a plurality of different homopolymer lengths and nucleotides: For each aligned/mapped sequence, a data array may be populated that comprises (i) the called homopolymers (e.g., A 1-mers, C 2-mers, etc.), (ii) the reference homopolymers (e.g., A 1-mers, C 2-mers, etc.), (iii) corresponding intensities, and (iv) corresponding lower/upper homopolymer boundary perturbations relative to an ideal homopolymer intensity threshold. For example, if 2-mers should in theory have a measured intensity of 200 with a lower cut-off of 150and an upper cut-off of 250, a particular 2-mer having been called with an intensity value of 230would have an upper perturbation of −20. (Of course, such a numbering is only an example and different scaling/numbering could also be used). Then, for each flow among the nucleotide flows, a data array comprising counts for the nucleotide species, called homopolymer, reference homopolymer, intensities, and perturbation may be generated. Such counts may be converted to frequencies, which may be used to generate probability distributions.

In an embodiment, a random training sample of reads (e.g., about 1 million reads, about 2 million reads, or more) may be obtained and aligned to a reference genome. The size of the training sample may vary according to experimental needs and objectives, with largest sizes allowing improved determination of parameters (and non-parametric thresholds). However, larger training samples typically lead to increases in required computational resources and/or time. The aligned reads may be post-processed to determine the joint distribution of true homopolymer length and observed flow signals for each nucleotide. The distributions may be further processed to generate accuracy values, which may then be used for recalibration of all reads produced by a base caller. The accuracy values may be presented as a graph of accuracy as a function of intensity (e.g., homopolymer length) for each flow signal. For a given flow signal, a graph of accuracy (which can sometimes be referred to as a flow quality value) may be related to homopolymer-calling error probabilities and generated using an expression −10×log₁₀ (1−Cₙ/C), where Cₙ represents a frequency of the homopolymer length n with highest frequency among homopolymers of lengths l, . . . , j observed in some interval of intensities, and where C=C₁+ . . . +Cⱼ represents the total of frequencies of homopolymers l, . . . , j observed with frequencies Cᵢ, . . . , Cⱼ, where i and j represent homopolymer lengths. Of course, such a graph may be represented in the form of a data array or table. The distributions may be determined separately for a plurality of regions (e.g., four quadrants) on an array/chip and for a plurality of bins of nucleotide flows (e.g., the first half of the flows making up a first bin, with the second half making up a second bin). In an example, such an accuracy graph or table may thus have 32 sets of accuracy graphs or table and their related homopolymer distributions, corresponding to the 4 nucleotide types, 4 regions out of 2-by-2 chip spatial stratification, and 2 partitions of flows. In other embodiments, spatial and flow partitions may be defined more or less densely.

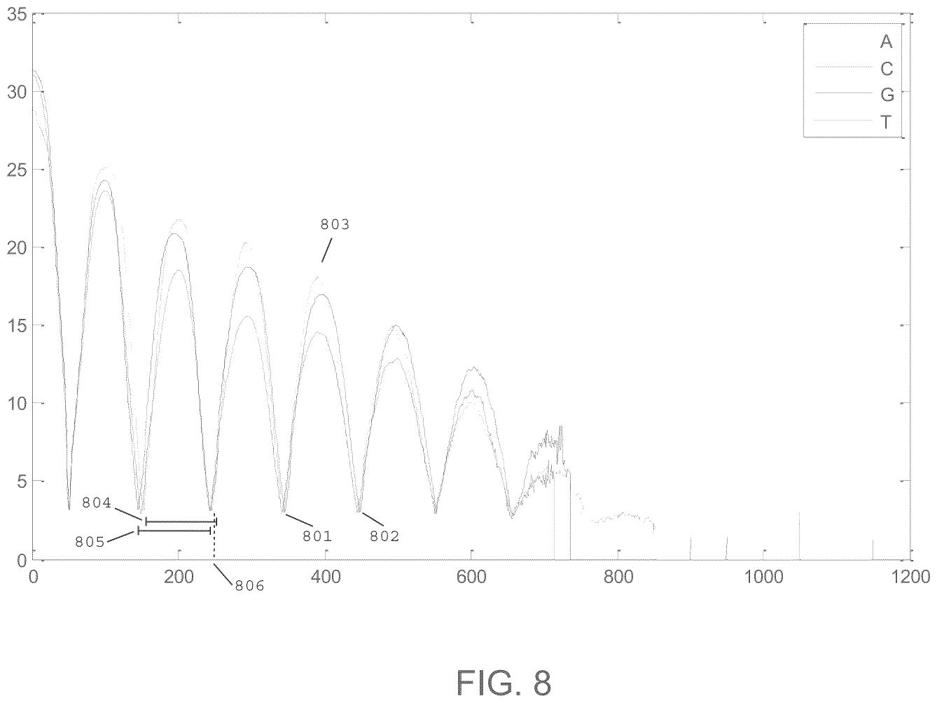

FIG. 8 illustrates a plot of accuracy as a function of intensities showing examples of recalibration thresholds. The x-axis represents (normalized) measured intensities. The y-axis shows accuracy determined using homopolymer-calling error probabilities as described above. Four accuracy curves are shown, one for each of nucleotides A, C, G, and T. In theory, each of the series of peaks should be centered around values such as i×100, where i =0, 1, 2, . . . , to represent 0-mers, 1-mers, 2-mers, etc. (Of course, such a numbering is only an example and different scaling/numbering could also be used). The local minima between the peaks mark thresholds between successive homopolymer peaks. For example, element 801 shows lower intensity thresholds for 4-mers of A, C, G, and T (which vary slightly according to the nucleotide), and element 802 shows upper intensity thresholds for 4-mers of A, C, G, and T (which vary slightly according to the nucleotide). Element 803 shows accuracy peaks for 4-mers of A, C, G, and T (which also vary slightly according to the nucleotide). The intensity values corresponding to the local minima can be determined from the graph using any suitable data analysis or curve analysis method known in the art, including smoothing, fitting, etc. In turn, successive local minima, once determined, may be used to define intervals corresponding to a given homopolymer length and nucleotide. For example, elements 804 and 805 show approximations of two such intervals for 2-mers for illustrative purposes. Interval 804 is slightly to the right of interval 805, reflecting a slight translation to the right in the accuracy curve for nucleotide A here relative to the curve of the other nucleotides (which are not exactly the same but are relatively clustered together left of the curve for A). Such intervals, once defined through their lower and upper endpoints, may be used to recalibrate some homopolymers. For example, suppose a called A 2-mer with the intensity marked by the dotted line 806 were being considered for potential recalibration. Given that interval 804 includes the intensity of dotted line 806, that call would remain unchanged. If the homopolymer had been called a C, G, or T 2-mer instead, however, recalibration to a 3-mer would probably be appropriate given that the intensity of dotted line 806 would be outside the allowed interval for a 2-mer. Finally, it should be noted that depending on the amounts of data available, the accuracy curves for certain long homopolymers may not always be dense enough to allow for determination of the corresponding local minima. For example, in FIG. 8 the peaks around intensities 700 and 800 begin to break down and peaks with higher intensities are not sufficiently populated. Using larger training sets would allow extension of the accuracy graph to higher homopolymers, however, there is a trade-off as such larger data sets would generally increase computational burden and analysis time.

### Parametric Recalibration

In an embodiment, the predicted intensity values may be obtained via a predictive model used by the base caller, which may be a model that can predict intensity values that would be likely to arise for candidate base call sequences given the underlying sequencing technology and operating parameters such as ordering of nucleotide flows, sensor characteristics, etc.). Using a predictive model, the measurement intensity values mᵢ₁, mᵢ₂, . . . , mᵢⱼ, . . . , m_{iM} represent a vector of measured values for M nucleotide flows associated with an i-th read (e.g., a set of normalized, calibrated values observed for the i-th read over the M flows) and the model-predicted intensity values pᵢ₁, pᵢ₂, . . . , pᵢⱼ, . . . , P_{iM} represent a vector of predicted values for the i-th read over the M flows under the predictive model. Such model-predicted base calling may be performed as described in Davey et al., U.S. Pat. Appl. Publ. No. 2012/0109598, published on May 3, 2012, and/or Sikora et al., U.S. Pat. Appl. Publ. No. 2013/0060482, published on Mar. 7, 2012, which are all incorporated by reference herein in their entirety. For recalibration, the measurements and model-predicted values may be obtained for each aligned read and for each nucleotide flow. Once the measurements and model-predicted values have been accumulated, the parameters a and b of a linear transformation (e.g., m=a×p+b) may be estimated to minimize, for certain sets of measurements/predictions corresponding to homopolymers of given length and nucleotide, a difference between them. The parameters of the linear transformation may be determined using any suitable data analysis or optimization method known in the art. In an embodiment, the parameters may be determined by solving under least-squares.

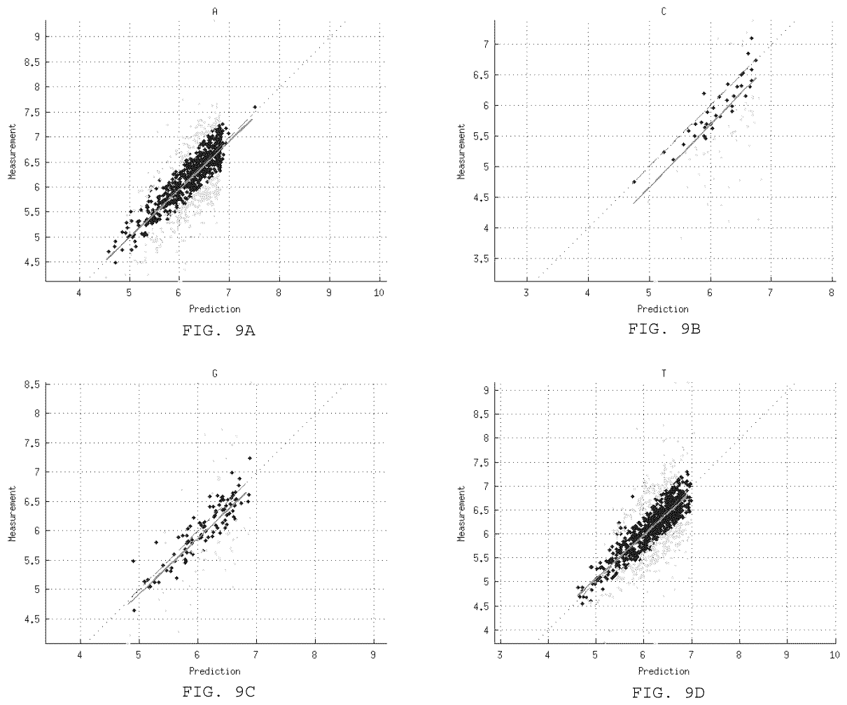

FIGS. 9A-9D illustrate exemplary plots of measurement/prediction clusters. The x-axis and y-axis respectively represent model-predicted and measured values for 7-mers of nucleotides A (FIG. 9A), C (FIG. 9B), G (FIG. 9C), and T (FIG. 9D) from a training set. Here, the model-predicted values are based on predictions for the reference homopolymer (post-alignment) rather than the homopolymer as initially determined by the base caller. The black points represent measurement/prediction pairs for base calls that were correct when compared against the reference. The gray points represent measurement/prediction pairs for base calls that were incorrect when compared against the reference. The dotted line represents the diagonal, which represents equality between measurement and prediction. The straight line segment represents the trend of prediction after recalibration, which is a linear representation of the a and b parameters obtained by optimization (e.g., least-squares). In an embodiment, the a and b parameters may be obtained by least-squares optimization of measurement/prediction pairs for base calls from the training subset that were correct when compared against the reference. In another embodiment, the a and b parameters could be obtained by least-squares optimization of measurement/prediction pairs for base calls from the training subset that were either correct or incorrect when compared against the reference.

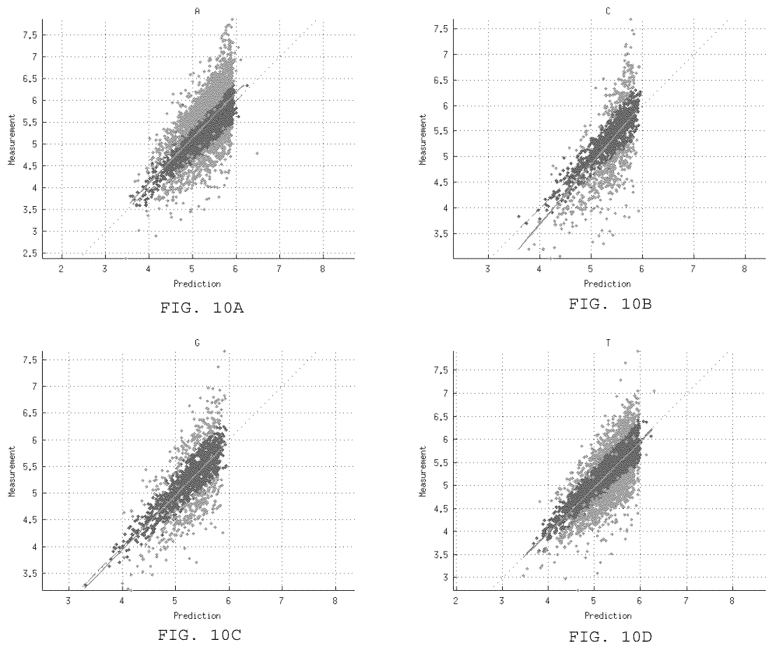

FIGS. 10A-10D illustrate exemplary plots of measurement/prediction clusters. The x-axis and y-axis are of a form similar to that of FIGS. 9A-9B and respectively represent model-predicted and measured values for 6-mers and nucleotides A (FIG. 10A), C (FIG. 10B), G (FIG. 10C), and T (FIG. 10D) and a subset of earlier flows (here, flows 1-330) from a training set. FIGS. 10A and 10B show measurement spikes on the upper right, a phenomenon likely to affect accuracy.

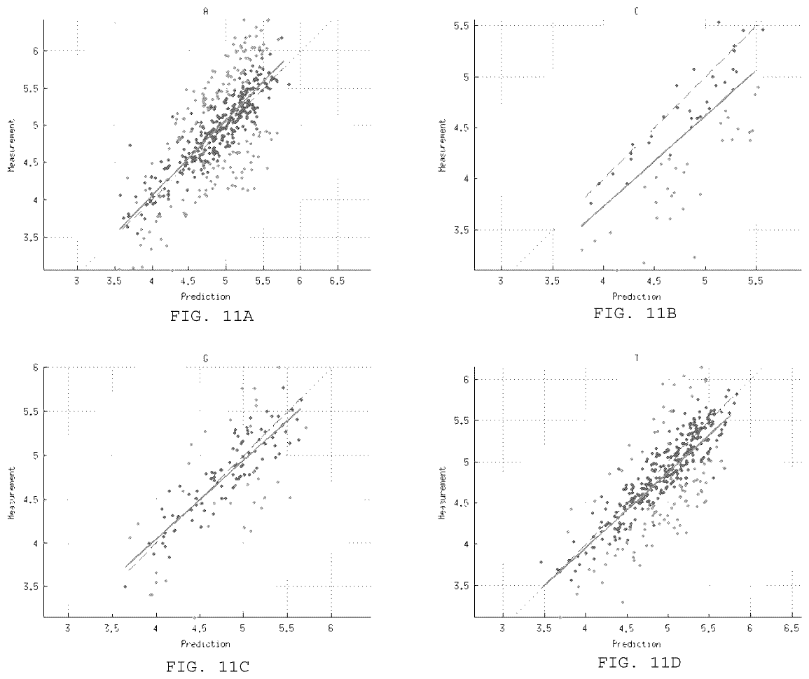

FIGS. 11A-11D illustrate exemplary plots of measurement/prediction clusters. The x-axis and y-axis are of a form similar to that of FIGS. 9A-9B and respectively represent model-predicted and measured values for 6-mers and nucleotides A (FIG. 11A), C (FIG. 11B), G (FIG. 11C), and T (FIG. 11D) and a subset of curlier flows (here, flows 331-660) from a training set. FIGS. 11A-11D do not show measurement spikes, illustrating that different partitions of flows can have different properties.

In an embodiment, a phenomenon such as the measurement spikes of FIGS. 10A and 10B may sometimes be addressed or mitigated by estimating the slope parameter a by least-squares optimization of measurement/prediction pairs taken only for base calls from the training subset that were correct when compared against the reference, while estimating the offset parameter b by least-squares optimization of measurement/prediction pairs for base calls from the training subset that were either correct or incorrect when compared against the reference. Such an approach is generally more robust, although typically would increase computational burden and analysis time.

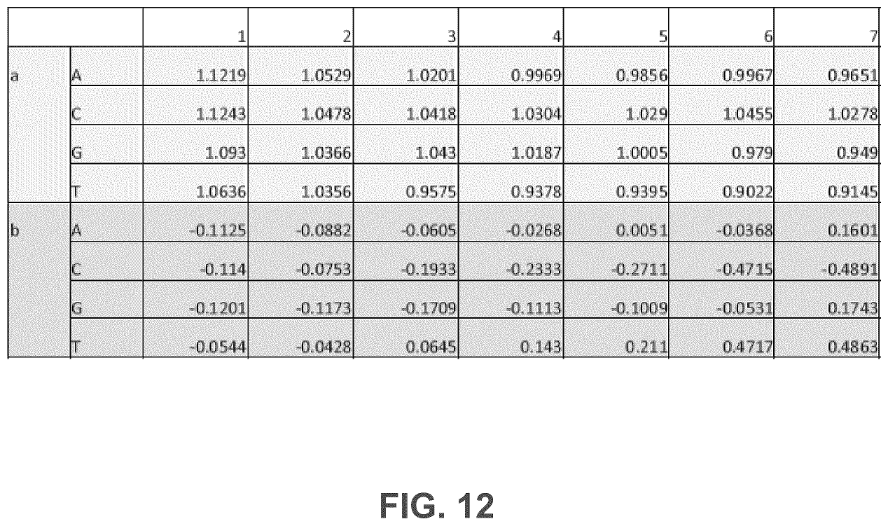

FIG. 12 shows a table with an example of recalibration model parameters. Shown are exemplary parameters a and b for homopolymers of lengths 1 through 7 and nucleotides A, C, G, and T. Linear parameters, such as shown in the table of FIG. 12, may be used for recalibration of model-predicted intensity values as described herein. In some cases, recalibration using linear parameters may be limited to homopolymers of at least a certain length, in which case a may be set to a value of 1 and b may be set to a value of 0. Also, although this particular table does not extend beyond length 7, parameters for longer homopolymers could also be obtained (possibly up to some predetermined maximum length that would typically be set according to the amount of data).

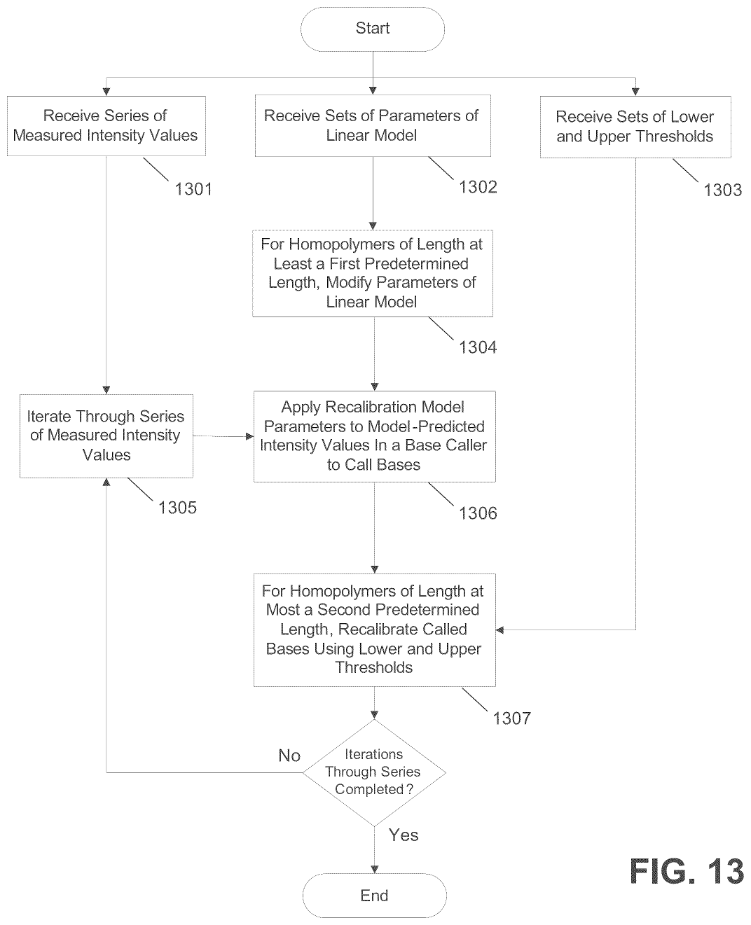

FIG. 13 illustrates an exemplary method for recalibrating sequencing data. In step 1301, a server or other computing means or resource receives a plurality of series of measured intensity values as described herein, for example. In step 1302, the server or other computing means or resource receives sets of parameters of a linear model for a plurality of different homopolymers and nucleotides, which may have been obtained using methods described herein, for example. In step 1303, the server or other computing means or resource receives sets of lower and upper thresholds for a plurality of different homopolymers and nucleotides, which may have been obtained using methods described herein. In step 1304, the server or other computing means or resource modifies, for homopolymers of length at least a first predetermined length, the parameters of a linear model. The first predetermined length may be factory pre-defined or user-specified. For example, if the first predetermined length is four then homopolymers of length less than four could be modified to have their a parameter reset to a value of 1 and their b parameter reset to a value of 0. In step 1305, the server or other computing means or resource iterates through each of the plurality of received series of measured intensity values. In step 1306, the server or other computing means or resource applies the linear model recalibration parameters to the model-predicted intensity values in a base caller as discussed herein to call bases. In step 1307, the server or other computing means or resource recalibrates, for homopolymers of length at most a second predetermined length, called bases using the received lower and upper thresholds. The second predetermined length may be factory pre-defined or user-specified. In an embodiment, homopolymers may be recalibrated by determining whether their intensity value falls within the lower and upper intensity value thresholds determined for the homopolymer. For example, if a 2-mer has been called for an intensity of 235, but that intensity would fall outside and above the range delimited by the lower and upper intensity value thresholds determined for 2-mers of the relevant nucleotide, the 2-mer may be recalibrated as a 3-mer, as discussed above in the context of FIG. 8.

In some embodiments, in addition to recalibrating homopolymers (and thus base sequences), related data or signals may also be modified consistently. For example, predicted base quality score may be adjusted by ensuring production of the same number of quality scores as the number of recalibrated base calls. In an embodiment, in the case of a deletion, the first predicted quality score may be removed; in the case of an insertion of a longer homopolymer, the last quality score of the homopolymer may be re-used; and in the case of insertion of a new 1-mer, a flow quality value may be used. In addition, a corresponding measured intensity may be modified for possible downstream analysis. For example, a measured intensity may be modified using the following expression: 98×(m−LT)/(UT−LT)+n×100−49, where n is the calibrated homopolymer length (before recalibration) and m is the intensity to be modified. (Of course, such constants or multipliers are only an example and different scaling/numbering could also be used.) For example, a 2-mer with intensity of 235 may be recalibrated to 3-mer if the lower and upper thresholds for 2-mers are 131 and 230, respectively, in which case the corresponding intensity may be modified as 98×(235−131)/(230−131)+2×100−49=254 (rounded to nearest integer). The same equation would keep the intensity unchanged if the lower and upper thresholds were identical to those one might expect in theory (e.g., 151 and 249, under an assumption that n-mers would be centered around intensities n×100 and separated at midpoints therebetween): 98×(235−151)/(249−151)+2×100−49=235.

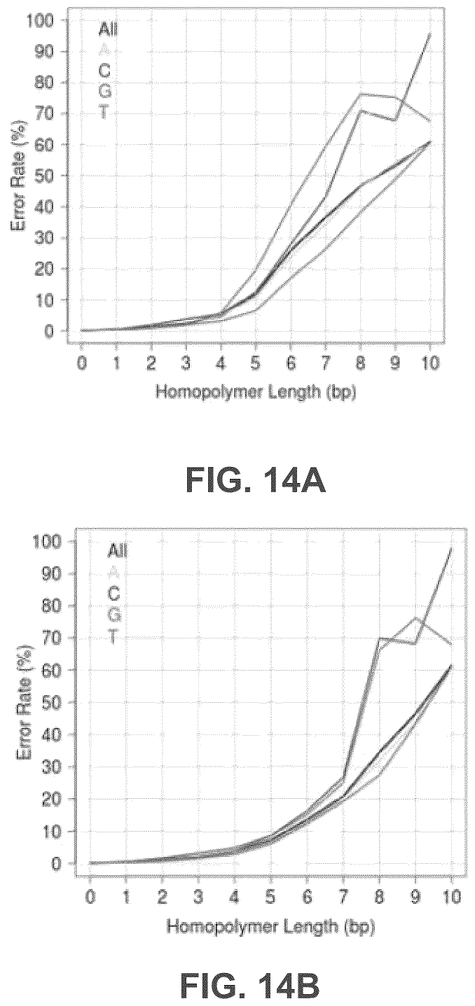

FIGS. 14A and 14B illustrate exemplary plots of error rate as a function of homopolymer length before and after recalibration. As illustrated, the error rates here are reduced significantly especially for homopolymer lengths between 5 and 8. FIG. 14B shows the error rate after recalibration including non-parametric recalibration for homopolymers of length at most 4 and parametric model recalibration for homopolymers of lengths 5 through 7. Of note, the parametric model recalibration may be used for longer homopolymers as well, at the cost of additional parameter estimation, which in turn may require use of a larger training data set. It should also be noted, however, that error rate reduction can vary depending on the complexity of the reference genome or sequence.

According to an exemplary embodiment, there is provided a method for improving base calling accuracy in nucleic acid sequencing, comprising: (a) exposing a plurality of template polynucleotide strands, sequencing primers, and polymerase disposed in a plurality of defined spaces disposed on a sensor array to a series of flows of nucleotide species according to a predetermined order; (b) obtaining a plurality of series of measured intensity values corresponding to the series of flows of nucleotide species and to the plurality of defined spaces disposed on the sensor array and randomly selecting a training subset of the plurality of series of measured intensity values; (c) generating a first plurality of series of base calls corresponding to the training subset of the plurality of series of measured intensity values using a base caller and aligning the first plurality of series of base calls to a reference genome or sequence using an aligner; (d) determining a plurality of intensity value thresholds corresponding to different homopolymer lengths and nucleotide species, and a plurality of parameters of a linear transformation corresponding to different homopolymer lengths and nucleotide species; (e) generating a second plurality of series of base calls corresponding to the plurality of series of measured intensity values using the base caller and, for homopolymers of a least a first predetermined length, at least some of the plurality of parameters of a linear transformation; and (f) recalibrating the second plurality of series of base calls corresponding to the plurality of series of measured intensity values, for homopolymers of at most a second predetermined length, using at least some of the plurality of intensity value thresholds.

In such a method, the plurality of intensity value thresholds may comprise a lower intensity threshold and an upper intensity threshold for each of the different homopolymer lengths and nucleotide species. The plurality of intensity value thresholds may comprise a set of lower intensity thresholds and upper intensity thresholds for each of nucleotide species A, C, G, and T determined using a graph of accuracy as a function of signal intensity. The accuracy may determined using an expression −10×log₁₀(1−Cₙ/C), where Cₙ represents a frequency of the homopolymer length n with highest frequency among homopolymers of lengths l, . . . , j, and wherein C=C₁+ . . . +Cⱼ represent the total of frequencies of homopolymers of lengths 1, . . . j. The lower intensity thresholds and upper intensity thresholds may correspond to local minima of the graph of accuracy as a function of signal intensity for each of nucleotide species A, C, G, and T.

In such a method, recalibrating the second plurality of series of base calls may comprise replacing a homopolymer base call called for a measured intensity value falling outside a range defined by the lower intensity threshold and upper intensity threshold for the homopolymer length and nucleotide species of the homopolymer base call with a different homopolymer base call. Recalibrating the second plurality of series of base calls may further comprise correcting the measured intensity value corresponding to the replaced homopolymer base call using an expression comprising a constant multiplied by a ratio between (i) a difference between the measured intensity value and a lower intensity threshold and (ii) a difference between an upper intensity threshold and a lower intensity threshold. The plurality of intensity value thresholds may comprise a plurality of separate sets of intensity value thresholds, each corresponding to a partition of the sensor array. The plurality of intensity value thresholds may comprise a plurality of separate sets of intensity value thresholds, each corresponding to a partition of the series of flows of nucleotide species. The plurality of intensity value thresholds may comprise a plurality of separate sets of intensity value thresholds, each corresponding to a partition of the sensor array and a partition of the series of flows of nucleotide species.

In such a method, the base caller may be configured to call bases at least in part using differences between the measured intensity values and model-predicted intensity values obtained using a predictive model of intensities responsive to flows of nucleotide species. The plurality of parameters of a linear transformation may comprise a slope and an offset for different homopolymer lengths and nucleotide species that represent a compensation for differences between measured intensity values and model-predicted intensity values. The plurality of parameters of a linear transformation may comprise parameters a and b for different homopolymer lengths and nucleotide species that minimize an absolute value of a difference between (i) a times the model-predicted intensity values plus b, and (ii) the measured intensity values. The plurality of parameters of a linear transformation may comprise a plurality of separate sets of parameters of a linear transformation, each corresponding to a partition of the sensor array. The plurality of parameters of a linear transformation may comprise a plurality of separate sets of parameters of a linear transformation, each corresponding to a partition of the series of flows of nucleotide species. The plurality of parameters of a linear transformation may comprise a plurality of separate sets of parameters of a linear transformation, each corresponding to a partition of the sensor array and a partition of the series of flows of nucleotide species. Generating the second plurality of series of base calls corresponding to the plurality of series of measured intensity values may comprise applying the plurality of parameters of a linear transformation to the model-predicted intensity values. Generating the second plurality of series of base calls corresponding to the plurality of series of measured intensity values may comprise transforming the model-predicted intensity values using the plurality of parameters of a linear transformation.

According to an exemplary embodiment, there is provided a non-transitory machine-readable storage medium comprising instructions which, when executed by a processor, cause the processor to perform a method for improving base calling accuracy in nucleic acid sequencing, comprising: (a) exposing a plurality of template polynucleotide strands, sequencing primers, and polymerase disposed in a plurality of defined spaces disposed on a sensor array to a series of flows of nucleotide species according to a predetermined order; (b) obtaining a plurality of series of measured intensity values corresponding to the series of flows of nucleotide species and to the plurality of defined spaces disposed on the sensor array and randomly selecting a training subset of the plurality of series of measured intensity values; (c) generating a first plurality of series of base calls corresponding to the training subset of the plurality of series of measured intensity values using a base caller and aligning the first plurality of series of base calls to a reference genome or sequence using an aligner; (d) determining a plurality of intensity value thresholds corresponding to different homopolymer lengths and nucleotide species, and a plurality of parameters of a linear transformation corresponding to different homopolymer lengths and nucleotide species; (c) generating a second plurality of series of base calls corresponding to the plurality of series of measured intensity values using the base caller and, for homopolymers of a least a first predetermined length, at least some of the plurality of parameters of a linear transformation; and (f) recalibrating the second plurality of series of base calls corresponding to the plurality of series of measured intensity values, for homopolymers of at most a second predetermined length, using at least some of the plurality of intensity value thresholds.

According to an exemplary embodiment, there is provided a system for improving base calling accuracy in nucleic acid sequencing, including: a plurality of template polynucleotide strands, sequencing primers, and polymerase disposed in a plurality of defined spaces disposed on a sensor array; an apparatus configured to expose the plurality of template polynucleotide strands, sequencing primers, and polymerase to a series of flows of nucleotide species according to a predetermined order; a machine-readable memory; and a processor configured to execute machine-readable instructions, which, when executed by the processor, cause the system to perform a method for improving base calling accuracy in nucleic acid sequencing, comprising: (a) obtaining a plurality of series of measured intensity values corresponding to the series of flows of nucleotide species and to the plurality of defined spaces disposed on the sensor array and randomly selecting a training subset of the plurality of series of measured intensity values; (b) generating a first plurality of series of base calls corresponding to the training subset of the plurality of series of measured intensity values using a base caller and aligning the first plurality of series of base calls to a reference genome or sequence using an aligner; (c) determining a plurality of intensity value thresholds corresponding to different homopolymer lengths and nucleotide species, and a plurality of parameters of a linear transformation corresponding to different homopolymer lengths and nucleotide species; (d) generating a second plurality of series of base calls corresponding to the plurality of series of measured intensity values using the base caller and, for homopolymers of a least a first predetermined length, at least some of the plurality of parameters of a linear transformation; and (c) recalibrating the second plurality of series of base calls corresponding to the plurality of series of measured intensity values, for homopolymers of at most a second predetermined length, using at least some of the plurality of intensity value thresholds.

According to an exemplary embodiment, there is provided a method for determining recalibration thresholds and parameters in nucleic acid sequencing, comprising: (a) exposing a plurality of template polynucleotide strands, sequencing primers, and polymerase disposed in a plurality of defined spaces disposed on a sensor array to a series of flows of nucleotide species according to a predetermined order; (b) obtaining a plurality of series of measured intensity values corresponding to the series of flows of nucleotide species and to the plurality of defined spaces disposed on the sensor array and randomly selecting a training subset of the plurality of series of measured intensity values; (c) generating a first plurality of series of base calls corresponding to the training subset of the plurality of series of measured intensity values using a base caller and aligning the first plurality of series of base calls to a reference genome or sequence using an aligner; and (d) determining a plurality of intensity value thresholds corresponding to different homopolymer lengths and nucleotide species, and a plurality of parameters of a linear transformation corresponding to different homopolymer lengths and nucleotide species.

According to an exemplary embodiment, there is provided a method for improving base call accuracy using recalibration thresholds and parameters, comprising: (a) receiving a plurality of intensity value thresholds corresponding to different homopolymer lengths and nucleotide species, and a plurality of parameters of a linear transformation corresponding to different homopolymer lengths and nucleotide species; (b) generating a plurality of series of base calls corresponding to a plurality of series of measured intensity values using a base caller and, for homopolymers of a least a first predetermined length, at least some of the plurality of parameters of a linear transformation; and (c) recalibrating the plurality of series of base calls corresponding to the plurality of series of measured intensity values, for homopolymers of at most a second predetermined length, using at least some of the plurality of intensity value thresholds. In such a method, (i) the plurality of intensity value thresholds and plurality of parameters may have been generated using an initial plurality of series of base calls corresponding to a randomly selected training subset of the plurality of series of measured intensity values; (ii) the plurality of series of measured intensity values may have been obtained as a result of a series of flows of nucleotide species to a sensor array comprising a plurality of defined spaces in which template polynucleotide strands, sequencing primers, and polymerase have been disposed; and (iii) the initial plurality of series of base calls may have been obtained using a base caller and aligned to a reference genome or sequence using an aligner.

According to an exemplary embodiment, there is provided a method for improving base calling accuracy in nucleic acid sequencing, comprising: exposing template polynucleotide strands, sequencing primers, and polymerase to flows of nucleotide species; obtaining a series of measured intensity values and randomly selecting a training subset therefrom; generating series of base calls using a base caller and aligning the series of base calls to a reference genome or sequence using an aligner; determining intensity value thresholds and parameters of a linear transformation corresponding to different homopolymer lengths and nucleotide species; generating series of base calls corresponding to the series of measured intensity values using at least some of the parameters of a linear transformation; and recalibrating the series of base calls corresponding to the plurality of series of measured intensity values using at least some of the intensity value thresholds.

Unless otherwise specifically designated herein, terms, techniques, and symbols of biochemistry, cell biology, genetics, molecular biology, nucleic acid chemistry, nucleic acid sequencing, and organic chemistry used herein follow those of standard treatises and texts in the relevant field.

Although the present description described in detail certain embodiments, other embodiments are also possible and within the scope of the present invention. For example, those skilled in the art may appreciate from the present description that the present teachings may be implemented in a variety of forms, and that the various embodiments may be implemented alone or in combination. Variations and modifications will be apparent to those skilled in the art from consideration of the specification and figures and practice of the teachings described in the specification and figures, and the claims.

1. A method for base calling in nucleic acid sequencing, comprising:
(a) exposing a plurality of template polynucleotide strands, sequencing primers, and polymerase disposed in a plurality of defined spaces disposed on a corresponding plurality of sensors of a sensor array to a series of flows of nucleotide species according to a predetermined order;
(b) obtaining a plurality of series of measured intensity values corresponding to the series of flows of nucleotide species and to the plurality of defined spaces disposed on the sensor array and randomly selecting a training subset of the plurality of series of measured intensity values;
(c) generating a first plurality of series of base calls corresponding to the training subset of the plurality of series of measured intensity values using a base caller and aligning the first plurality of series of base calls to a reference genome using an aligner;
(d) determining a plurality of intensity value thresholds corresponding to different homopolymer lengths and nucleotide species, and a plurality of parameters of a linear transformation relating model-predicted intensity values and the measured intensity values corresponding to different homopolymer lengths and nucleotide species;
(e) generating a second plurality of series of base calls corresponding to the plurality of series of measured intensity values using a first recalibration and a second recalibration;
(f) for homopolymers of a least a first predetermined length, applying the first recalibration, the first recalibration using at least some of the plurality of parameters of a linear transformation to form first recalibrated homopolymer base calls, corresponding to the homopolymers having at least the first predetermined length, for the second plurality of series of base calls; and
(g) for homopolymers of at most a second predetermined length, applying the second recalibration to the base calls in the second plurality of series of base calls corresponding to the plurality of series of measured intensity values, the second recalibration using at least some of the plurality of intensity value thresholds to form second recalibrated homopolymer base calls, corresponding to the homopolymers having at most the second predetermined length, for the second plurality of series of base calls.

2. The method of claim 1, wherein the plurality of intensity value thresholds comprises a lower intensity threshold and an upper intensity threshold for each of the different homopolymer lengths and nucleotide species.

3. The method of claim 1, wherein the plurality of intensity value thresholds comprises a set of lower intensity thresholds and upper intensity thresholds for each of nucleotide species A, C, G, and T determined using a graph of accuracy as a function of signal intensity.

4. The method of claim 3, wherein the accuracy is determined using an expression −10×log10(1−Cn/C), where Cn represents a frequency of the homopolymer length n with highest frequency among homopolymers of lengths l, . . . , j, and wherein C=C1+ . . . +Cj represent the total of frequencies of homopolymers of lengths l, . . . , j.

5. The method of claim 3, wherein the lower intensity thresholds and upper intensity thresholds correspond to local minima of the graph of accuracy as a function of signal intensity for each of nucleotide species A, C, G, and T.

6. The method of claim 1, wherein the plurality of intensity value thresholds comprises a plurality of separate sets of intensity value thresholds, each corresponding to a partition of the sensor array.

7. The method of claim 1, wherein the plurality of intensity value thresholds comprises a plurality of separate sets of intensity value thresholds, each corresponding to a partition of the series of flows of nucleotide species.

8. The method of claim 1, wherein the plurality of intensity value thresholds comprises a plurality of separate sets of intensity value thresholds, each corresponding to a partition of the sensor array and a partition of the series of flows of nucleotide species.

9. The method of claim 1, wherein the base caller is configured to call bases at least in part using differences between the measured intensity values and the model-predicted intensity values obtained using a predictive model of intensities responsive to flows of nucleotide species.

10. The method of claim 9, wherein the plurality of parameters of a linear transformation comprises a slope and an offset for different homopolymer lengths and nucleotide species that represent a compensation for differences between the measured intensity values and the model-predicted intensity values.

11. The method of claim 9, wherein the plurality of parameters of a linear transformation comprises parameters a and b for different homopolymer lengths and nucleotide species that minimize an absolute value of a difference between (i) a times the model-predicted intensity values plus b, and (ii) the measured intensity values.

12. The method of claim 1, wherein the plurality of parameters of a linear transformation comprises a plurality of separate sets of parameters of a linear transformation, each corresponding to a partition of the sensor array.

13. The method of claim 1, wherein the plurality of parameters of a linear transformation comprises a plurality of separate sets of parameters of a linear transformation, each corresponding to a partition of the series of flows of nucleotide species.

14. The method of claim 1, wherein the plurality of parameters of a linear transformation comprises a plurality of separate sets of parameters of a linear transformation, each corresponding to a partition of the sensor array and a partition of the series of flows of nucleotide species.

15. The method of claim 9, wherein applying the first recalibration comprises applying the plurality of parameters of a linear transformation to the model-predicted intensity values.

16. The method of claim 15, wherein applying the first recalibration comprises transforming the model-predicted intensity values using the plurality of parameters of a linear transformation.

17. The method of claim 2, wherein applying the second recalibration to the base calls in the second plurality of series of base calls comprises replacing a homopolymer base call called for a measured intensity value falling outside a range defined by the lower intensity threshold and the upper intensity threshold for the homopolymer length and nucleotide species of the homopolymer base call with a different homopolymer base call for the second plurality of series of base calls.

18. The method of claim 17, wherein applying the second recalibration to the base calls in the second plurality of series of base calls further comprises correcting the measured intensity value corresponding to the replaced homopolymer base call using an expression comprising a constant multiplied by a ratio between (i) a difference between the measured intensity value and the lower intensity threshold and (ii) a difference between the upper intensity threshold and the lower intensity threshold.

19. A non-transitory machine-readable storage medium comprising instructions which, when executed by a processor, cause the processor to perform a method for base calling in nucleic acid sequencing, comprising:
(a) obtaining a plurality of series of measured intensity values corresponding to a series of flows of nucleotide species and to a plurality of defined spaces disposed on a corresponding plurality of sensors of a sensor array of a nucleic acid sequencing device, and randomly selecting a training subset of the plurality of series of measured intensity values, wherein the plurality of measured intensity values is produced by the nucleic acid sequencing device in response to exposing a plurality of template polynucleotide strands, sequencing primers, and polymerase disposed in the plurality of defined spaces disposed on the sensor array to the series of flows of nucleotide species according to a predetermined order;
(b) generating a first plurality of series of base calls corresponding to the training subset of the plurality of series of measured intensity values using a base caller and aligning the first plurality of series of base calls to a reference genome using an aligner;
(c) determining a plurality of intensity value thresholds corresponding to different homopolymer lengths and nucleotide species, and a plurality of parameters of a linear transformation relating model-predicted intensity values and the measured intensity values corresponding to different homopolymer lengths and nucleotide species;
(d) generating a second plurality of series of base calls corresponding to the plurality of series of measured intensity values using a first recalibration and a second recalibration;
(e) for homopolymers of a least a first predetermined length, applying the first recalibration, the first recalibration using at least some of the plurality of parameters of a linear transformation to form first recalibrated homopolymer base calls, corresponding to the homopolymers having at least the first predetermined length, for the second plurality of series of base calls; and
(f) for homopolymers of at most a second predetermined length, applying the second recalibration to the base calls in the second plurality of series of base calls corresponding to the plurality of series of measured intensity values, the second recalibration using at least some of the plurality of intensity value thresholds to form second recalibrated homopolymer base calls, corresponding to the homopolymers having at most the second predetermined length, for the second plurality of series of base calls.

20. A system for base calling in nucleic acid sequencing, including:
a plurality of template polynucleotide strands, sequencing primers, and polymerase disposed in a plurality of defined spaces disposed on a corresponding plurality of sensors of a sensor array;
an apparatus configured to expose the plurality of template polynucleotide strands, sequencing primers, and polymerase to a series of flows of nucleotide species according to a predetermined order;
a machine-readable memory; and
a processor configured to execute machine-readable instructions, which, when executed by the processor, cause the system to perform a method for base calling, comprising:
(a) obtaining a plurality of series of measured intensity values corresponding to the series of flows of nucleotide species and to the plurality of defined spaces disposed on the sensor array and randomly selecting a training subset of the plurality of series of measured intensity values;
(b) generating a first plurality of series of base calls corresponding to the training subset of the plurality of series of measured intensity values using a base caller and aligning the first plurality of series of base calls to a reference genome using an aligner;
(c) determining a plurality of intensity value thresholds corresponding to different homopolymer lengths and nucleotide species, and a plurality of parameters of a linear transformation relating model-predicted intensity values and the measured intensity values corresponding to different homopolymer lengths and nucleotide species;
(d) generating a second plurality of series of base calls corresponding to the plurality of series of measured intensity values using a first recalibration and a second recalibration;
(e) for homopolymers of a least a first predetermined length, applying the first recalibration, the first recalibration using at least some of the plurality of parameters of a linear transformation to form first recalibrated homopolymer base calls, corresponding to the homopolymers having at least the first predetermined length, for the second plurality of series of base calls; and
(f) for homopolymers of at most a second predetermined length, applying the second recalibration to the base calls in the second plurality of series of base calls corresponding to the plurality of series of measured intensity values, the second recalibration using at least some of the plurality of intensity value thresholds to form second recalibrated homopolymer base calls, corresponding to the homopolymers having at most the second predetermined length, for the second plurality of series of base calls.
